(19) United States
(12) Patent Application Publication
(10) Pub. No.: US 2008/0051879 A1
(43) Pub. Date: Feb. 28, 2008

(54) METHODS OF TREATING VENOUS VALVE RELATED CONDITIONS WITH A FLOW-MODIFYING IMPLANTABLE MEDICAL DEVICE

(75) Inventors: Brian C. Case, Lake Villa, IL (US); Gary Bradford Shirley, Bloomington, IN (US); Ram H. Paul JR., Bloomington, IN (US); Jacob A. Flagle, New Palestine, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/844,003

(22) Filed: Aug. 23, 2007

Related U.S. Application Data
(60) Provisional application No. 60/839,605, filed on Aug. 23, 2006.
(60) Provisional application No. 60/858,166, filed on Nov. 10, 2006.

(51) Int. Cl. A61F 2/06

(52) U.S. Cl. 623/1.24

(57) ABSTRACT

Implantable medical devices adapted to modify fluid flow within a body vessel are provided herein. The medical devices may include a fluid flow restricting channel configured to reduce longitudinal fluid flow in a retrograde direction or in an antegrade direction. Preferably, the medical devices are flow-modifying devices that reduce fluid flow through the medical device to a greater extent in a retrograde direction than in an antegrade direction. Methods of treatment comprising the step of implanting a flow-modifying medical device within a body vessel are also provided. Flow-modifying devices are useful, for example, in treating venous valve related conditions.

### RELATED APPLICATIONS

This application claims the benefit of both U.S. provisional patent application Ser. No. 60/839,605, filed Aug. 23, 2006 and entitled, “Flow-Modifying Medical Devices,” by Shirley et al., and U.S. provisional patent application Ser. No. 60/858,166, filed Nov. 10, 2006 and entitled, “Implantable Valves with Flow-Modifying Surface,” by Shirley et al., both of which are incorporated by reference herein in their entirety.

### TECHNICAL FIELD

The present invention relates to methods of treating medical conditions by reducing fluid flow through a body vessel. For example, methods are provided for treating a venous-valve related condition by implanting a flow modifying medical device within a body vessel.

### BACKGROUND

Many vessels in animals transport fluids from one body location to another in a substantially unidirectional manner along the length of the vessel. Native valves within the heart and veins function to regulate blood flow within these body vessels. For example, heart valves direct the flow of blood into and out of the heart and to other organs, while venous valves direct the flow of blood toward the heart. Body vessels such as veins transport blood to the heart, while arteries carry blood away from the heart.

In many mammals, small semilunar valves known as “venous valves” (valvulae vienosa) are found within the extremity veins. Such venous valves function as one-way check valves to maintain the flow of venous return blood in the direction toward the heart, while preventing blood from backflowing in a direction away from the heart. Heart valves open and close 60 to 150 times per minute with pressures of up to 250 mm Hg. On the other hand, venous valves typically remain open with minimal forward flow and close with flow reversal. Reverse venous flow may develop intermittent pressures of 150 mm Hg. Venous valves are particularly important in the veins of the lower extremities, as venous blood returning from the lower extremities is required to move against a long hydrostatic column, especially when the subject animal is in a standing or upright position. Venous valves are typically bicuspid valves positioned at varying intervals within veins to permit substantially unidirectional blood to flow toward the heart. These natural venous valves open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. The venous valve leaflets, however, do not normally bend in the opposite direction and therefore return to a closed position to restrict or prevent blood flow in the opposite, i.e. retrograde, direction after the pressure is relieved. The venous valve leaflet structures, when functioning properly, extend radially inwardly toward one another such that the tips contact each other to restrict backflow of blood.

Venous valves, especially those in the upper leg, perform an important function. When a person rises from a seated to a standing position, arterial blood pressure increases instantaneously to insure adequate perfusion to the brain and other critical organs. In the legs and arms, the transit time of this increased arterial pressure is delayed, resulting in a temporary drop in venous pressure. The venous pressure in the feet of someone walking is of the order of 25 mmHg (3.3 kPa), whereas in the feet of an individual standing absolutely still it is of the order of 90 mmHg (12 kPa). Venous pressure drops as blood flow responds to body position change and gravity, thereby reducing the volume of blood available to the right heart and possibly reducing the flow of oxygenated blood to the brain. In such a case, a person could become light headed, dizzy or experience syncope. It is the function of valves in the iliac, femoral and, to a lesser degree, more distal vein valves to detect these drops in pressure and resulting change of direction of blood flow and to close to prevent blood from pooling in the legs to maintain blood volume in the heart and head. The valves reopen and the system returns to normal forward flow when the reflected arterial pressure again appears in the venous circulation. Compromised valves, however, would allow reverse blood flow and pooling.

Occasionally, congenital defects or injury to valves within a body vessel can result in an undesirable amount of retrograde fluid flow across a valve therein, and compromise the unidirectional flow of fluid across the valve. In the condition of venous valve insufficiency, the valve leaflets do not function properly. Incompetent venous valves can result in symptoms such as swelling of the legs or varicose veins, causing great discomfort and pain to the patient. If left untreated, venous valve insufficiency can result in excessive retrograde venous blood flow through incompetent venous valves, which can cause venous stasis ulcers of the skin and subcutaneous tissue. Venous valve insufficiency can occur in the superficial venous system, such as the saphenous veins in the leg, or in the deep venous system, such as the femoral and popliteal veins extending along the back of the knee to the groin. Chronic venous insufficiency arises from long duration venous hypertension caused by valvular insufficiency and/or venous obstruction secondary to venous thrombosis. Other primary causes of CVI include varicosities of long duration, venous hypoplasia and arteriovenous fistula. The signs and symptoms of CVI have been used to classify the degree of severity of the disease, and reporting standards have been published. Studies demonstrate that deterioration of venous hemodynamic status correlates with disease severity. Venous reflux, measured by ultrasound studies, is the method of choice of initial evaluation of patients with pain and/or swelling in the lower extremities. In most serious cases of CVI, venous stasis ulcers are indicative of incompetent venous valves in all systems, including superficial, common, deep and communicating veins. This global involvement affects at least 30% of all cases. Standard principles of treatment are directed at elimination of venous reflux. Based on this observation, therapeutic intervention is best determined by evaluating the extent of valvular incompetence, and the anatomical distribution of reflux. Valvular incompetence, a major component of venous hypertension, is present in about 60% of patients with a clinical diagnosis of CVI.

Various implantable medical devices are advantageously inserted within various body vessels, such as veins, to modify fluid flow. Minimally invasive techniques and catheter delivery systems for placement of intraluminal medical devices have been developed to treat and repair undesirable conditions within body vessels, including treatment of conditions that affect blood flow such as venous valve insufficiency. Various percutaneous methods of implanting medical devices within the body using intraluminal transcatheter delivery systems can be used to treat a variety of conditions. One or more intraluminal medical devices can be introduced to a point of treatment within a body vessel using a delivery catheter device passed through the vasculature communicating between a remote introductory location and the implantation site, and released from the delivery catheter device at the point of treatment within the body vessel. Intraluminal medical devices can be deployed in a body vessel at a point of treatment and the delivery device subsequently withdrawn from the vessel, while the medical device retained within the vessel can provide sustained improvement in valve function or increased vessel patency. For example, an implanted medical device can improve the function of native valves by blocking or reducing retrograde fluid flow. Alternatively, prosthetic valves can be implanted to replace the function of damaged or absent native valves within the body.

Medical conditions caused by incompetent venous valves have been treated by the percutaneous insertion of an endovascular prosthetic device such as a valve under fluoroscopic guidance. The device can be advanced to the desired intravascular location using guide wires and catheters. One challenge for development of prosthetic devices for implantation within the venous system is mitigating thrombus formation that can occlude the vessel and/or lead to loss of functionality of the valve structures that regulate blood flow. In contrast to the arterial system, the lower flow rates in the deep veins of the legs and feet can lead to stagnation of blood in the pockets about the bases of the leaflets or valve structure due to the inability of the blood to be flushed and refreshed thereabout. The pockets can fill with thrombus that compromises the ability of the leaflets or valve structure to open and close in response to antegrade and retrograde flow (i.e., pressure differentials across the valve). For example, fibrinogen absorbed on to the surface of an implanted prosthetic valve can form a layer that triggers the biochemical pathway leading fibrin deposition, platelet aggregation, and thrombus formation.

What is needed are implantable medical devices capable of desirably modifying fluid flow within a body vessel while maintaining fluid flow across the implanted device. Fluid flow modification can include permitting fluid to flow in a first direction with a lower resistance than in the opposite, retrograde direction, thereby enhancing or improving the function of one-way venous valves. Flow-modifying medical devices deliverable by minimally-invasive transcatheter techniques are particularly desirable.

### SUMMARY

Methods of treating various medical conditions, such as venous valve-related conditions, by modifying fluid flow through a body vessel are provided. The methods may include the endoluminal implantation of a means for regulating fluid flow within a body vessel such as a vein in a manner providing a greater resistance to fluid flow through the body vessel in a retrograde direction (i.e., away from the heart) than in an antegrade direction (i.e., toward the heart). For example, an implantable flow-modifying medical device may be implanted in a body vessel in communication with a vein. The flow-modifying medical devices are preferably configured to restrict the rate of fluid flow within the body vessel by about 0.5-30% when passing through the flow-modifying medical device.

The flow-modifying medical device is preferably configured to reduce the rate of fluid flow through a body vessel in a flow direction-dependent manner, preferably by reducing the rate of fluid flow in a first direction less than in a second direction. For example, the flow-modifying medical device may be adapted to reduce blood flow in an antegrade direction less than fluid flow in a retrograde direction. The flow-modifying medical device may be configured to provide a greater resistance to fluid flow across the medical device in the retrograde direction than in the antegrade reduction. For example, a fluid flow passing across the flow-modifying medical device in a retrograde direction may be reduced by about 0.5-20% more than the same rate and pressure of fluid flow in the opposite, antegrade direction.

The flow-modifying medical device may have various configurations, but desirably includes a fluid contact surface defining a bi-directional fluid flow restricting channel adapted to conduct fluid through the device along a longitudinal axis. The channel may narrow from each end to a minimum cross-sectional area defining an orifice positioned between an inlet having a first cross-sectional area and an outlet having a second cross-sectional area. Unless otherwise indicated, the cross-sectional areas described herein are measured perpendicular to the longitudinal axis. The orifice preferably has a cross-sectional area that is less than the first cross-sectional area and/or the second cross-sectional area. The orifice can have any suitable configuration, but has a substantially circular shape with a diameter of about 3 mm or more. The ratio between the first cross-sectional area at the inlet and the cross-sectional area of the orifice may be selected to provide a desired resistance to fluid flow in the antegrade direction. For example, the ratio between the first cross-sectional area at the inlet and the cross-sectional area of the orifice is desirably between about 1.0 and 5.0, and preferably between about 2.0 and 2.5. The second cross-sectional area at the outlet is preferably substantially equal to the first cross-sectional area at the inlet. The ratio between the second cross-sectional area at the outlet and the cross-sectional area of the orifice may be selected to provide a desired resistance to fluid flow in the retrograde direction, and may be about 1.0 and 5.0, preferably between about 2.0 and 2.5. The orifice may have a cross-sectional area that is less than the first cross-sectional area and less than the second cross-sectional area. In particular, the orifice may contain the longitudinal axis and is configured as a circle having a diameter configured to prevent or mitigate incidence of thrombotic stenosis of the orifice. Preferably, the orifice may have a diameter of at least 3 mm. The orifice may also have a diameter of greater than or less than 3 mm, including diameters of about 4, 5, 6 or 7 mm, or more. The ratio between the cross-sectional surface area of the inlet and the cross-sectional area of the orifice is preferably between about 1.0 and 5.0. Preferably, the inlet and the outlet each have a cross-sectional area of between about 75 mm² and 185 mm².

The fluid contact surface defining the fluid flow restricting channel through the flow-modifying medical device may have any suitable shape, but preferably has a modified “dumbbell” configuration including a narrow orifice between a wider inlet and outlet. An antegrade flow receiving surface may extend from the inlet to the orifice, and a retrograde flow receiving surface may extend from the outlet to the orifice. To provide a grater resistance to fluid flow in the retrograde direction than in the antegrade direction, the antegrade flow receiving surface may be shaped differently from the retrograde flow receiving surface. Desirably, the antegrade flow receiving surface has a frustoconical cross section in a first radial bisecting plane containing the longitudinal axis and the retrograde flow receiving surface preferably has an arcuate cross section in the first plane. The antegrade flow receiving surface may form an angle of about 20-70 degrees, preferably about 40-50 degrees, with respect to the wall of a body vessel upon implantation. In contrast, the retrograde flow receiving surface preferably has an arcuate surface around the orifice, forming a “bowl-like” cross section in the first radial bisecting plane. The arcuate surface preferably has a radius of curvature that is less than the diameter of a circular orifice (or longest distance across of a non-circular orifice), preferably about 50-80% of the diameter of the orifice. Increasing the radius of curvature of a curved surface of the retrograde receiving surface may increase the resistance to fluid flow in the retrograde direction through the fluid flow restricting channel.

### BRIEF DESCRIPTION OF THE DRAWINGS

FIG. 1A is side view of a first flow-modifying medical device formed from a tubular member.

FIG. 1B is a cross-sectional view of the first flow-modifying medical device in FIG. 1A.

FIG. 1C is a top end view of the first flow-modifying medical device of FIG. 1A and FIG. 1B.

FIG. 2 is a perspective view of a second flow-modifying medical device comprising fluid flow channel formed from a forming film attached to an implantable frame.

FIG. 3A is a cross-sectional view of a third flow-modifying medical device.

FIG. 3B is a cross-sectional view of a fourth flow-modifying medical device having an antegrade flow receiving surface at a first angle.

FIG. 3C is a cross-sectional view of a variation of the fourth flow-modifying medical device having an antegrade flow receiving surface at a second angle.

FIG. 4 is a cross-sectional view of a fifth flow-modifying medical device having an orifice comprising a channel between the antegrade flow receiving surface and the retrograde flow receiving surface.

FIG. 5A is a side view of a sixth flow-modifying medical device.

FIG. 5B is an end view of the fifth flow-modifying medical device shown in FIG. 5A.

FIG. 6 is side cutaway view of a flow modifying medical device in a radially compressed configuration within the distal end of a delivery catheter.

### DETAILED DESCRIPTION

The following detailed description and appended drawings describe and illustrate various exemplary embodiments of the invention. The description and drawings serve to enable one skilled in the art to make and use the invention. The preferred embodiments of the present invention described below relate particularly to methods for mitigating symptoms of venous valve incompetence. The methods of treatment preferably include implantation of a means for regulating fluid flow within a body vessel, such as a vein. In particular, such methods may include endoluminal implantation of a flow modifying device in a manner providing a greater resistance to fluid flow through the flow modifying device in a retrograde direction (i.e., away from the heart) than in an antegrade direction (i.e., toward the heart). While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

### DEFINITIONS

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response, and is distinguished from a mild, transient inflammation which typically accompanies surgery or implantation of foreign objects into a living organism.

As used herein, the term “body vessel” means any body passage lumen that conducts fluid, including but not limited to blood vessels such as veins or arteries.

The medical devices of the embodiments described herein may be oriented in any suitable absolute orientation with respect to a body vessel. The recitation of a “first” direction is provided as an example. Any suitable orientation or direction may correspond to a “first” direction. The medical devices of the embodiments described herein may be oriented in any suitable absolute orientation with respect to a body vessel. For example, the first direction can be a radial direction in some embodiments.

The terms “frame” and “support frame” are used interchangeably herein to refer to a structure that can be implanted, or adapted for implantation, within the lumen of a body vessel. The valve support frame can have any suitable configuration, but is preferably a radially expandable structure comprising a plurality of struts and bends and enclosing an interior lumen.

As used herein, “antegrade fluid flow” refers to the flow of fluid in a primary direction of normal movement within a body vessel. For example, in veins, antegrade fluid flow proceeds primarily toward the heart. As used herein, “retrograde fluid flow” refers to fluid flow in a direction opposite the primary (antegrade) direction of fluid flow. Retrograde flow in a vein is primarily directed away from the heart.

As used herein, “orifice” refers to an opening within a fluid conducting channel defined at least in part by an implantable medical device. Preferably, the orifice has a diameter configured to prevent or mitigate incidence of thrombotic stenosis of the orifice. For example, an orifice may have a diameter of at least 3 mm when the medical device is adapted for implantation within a blood vessel, so as to reduce the likelihood of thrombosis. The orifice may also have a diameter of greater than 3 mm, including diameters of about 4, 5, 6 or 7 mm, or more. The orifice is desirably structured to remain substantially open to permit fluid flow therethrough, and is preferably of a substantially invariant cross-sectional area upon implantation of the medical device. An orifice preferably has a smaller cross-sectional area than the inlet or outlet of a fluid flow channel in communication with the orifice. Typically, the orifice is the narrowest portion of the fluid flow channel through a flow-modifying device. The orifice can also be the line of intersection between an antegrade flow receiving surface and a retrograde flow receiving surface, which can face opposite longitudinal directions. While preferred embodiments provide medical devices with a single orifice, other embodiments include flow-modifying devices comprising two or more orifices extending between the antegrade flow receiving surface and the retrograde flow receiving surface.

The recitation of a “forming material” refers to any material suitable for defining the outline of the flow restricting channel. A forming material may comprise any material or combination of materials that are suitable for conducting fluid. Depending on the flow modifying properties desired, a forming material may be sufficiently rigid to maintain patency of the flow restricting channel during fluid flow. The forming material is preferably flexible enough to permit intralumenal delivery from a catheter within the body vessel and to prevent undesirable resistance to the movement of the body vessel (e.g., physiological changes in the diameter of the body vessel). A forming material may include one or more materials selected from the group consisting of a biocompatible polymer such as polyurethane or expanded tetrafluoroethylene (ePTFE), a tissue derived material such as an extracellular matrix material, and a composite material comprising multiple polymers and/or tissue derived materials.

As used herein, the terms “proximal” and “distal” describe longitudinal directions in opposing axial ends of a medical device, such as a flow-modifying medical device, and components thereof. The term “proximal” is used in its conventional sense to refer to the end of the device (or component) that is closest to the clinician conducting the implantation of the medical device from a delivery catheter inserted within a body vessel. The term “distal” is used in its conventional sense to refer to the end of the device (or component) that is initially farther from the clinician implanting the device with the delivery catheter.

Methods of Treatment

Various methods of treatment are provided. These methods may include altering fluid flow within a body vessel, for example to treat a venous-valve related condition. In particular, methods of altering fluid flow in a directionally-dependent manner are provided that may include the step of implanting a flow modifying medical device within a body vessel in a manner providing a higher resistance to fluid flow in a retrograde direction (i.e., away from the heart) than in an antegrade direction (i.e., toward the heart). The methods of treatment are exemplified by methods of treating a venous valve-related condition, although the methods may also be used to treat other medical conditions by modifying the resistance to fluid flow through a body vessel.

A “venous valve-related condition” is any condition presenting symptoms that can be diagnostically associated with improper function of one or more venous valves. In mammalian veins, venous valves are positioned along the length of the vessel in the form of leaflets disposed annularly along the inside wall of the vein which open to permit blood flow toward the heart and close to prevent back flow. These venous valves open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. The leaflets, however, do not normally bend in the opposite direction and therefore return to a closed position to restrict or prevent blood flow in the opposite, i.e. retrograde, direction after the pressure is relieved. The leaflets, when functioning properly, extend radially inwardly toward one another such that the tips contact each other to block backflow of blood. Two examples of venous valve-related conditions are chronic venous insufficiency and varicose veins.

The human venous system of the lower limbs consists essentially of the superficial venous system and the deep venous system with perforating veins connecting the two systems. The superficial system includes the long or great saphenous vein and the short saphenous vein. The deep venous system includes the anterior and posterior tibial veins which unite to form the popliteal vein, which in turn becomes the femoral vein when joined by the short saphenous vein. Venous blood flow returns de-oxygenated blood from the distal extremities to the heart via two mechanisms. The first is the perfusion pressure resulting from the arterial blood flow through tissue to the venous circulation system. Where arterial pressure prior to perfusion may be 60 to 200 mm Hg, the resulting venous pressure is typically 10 to 40 mm Hg. The second mechanism is the calf muscle, which, when contracted, compresses the veins (tibial and peroneal) overlying the bone and, through a system of valves, directs blood flow toward the heart. This is the organized flow of blood through a normal, healthy person.

In the condition of venous valve insufficiency, the valve leaflets do not function properly. For example, the vein can be too large in relation to the leaflets so that the leaflets cannot come into adequate contact to prevent backflow (primary venous valve insufficiency), or as a result of clotting within the vein that thickens the leaflets (secondary venous valve insufficiency). Incompetent venous valves can result in symptoms such as limb swelling and varicose veins, causing great discomfort and pain to the patient. If left untreated, venous valve insufficiency can result in excessive retrograde venous blood flow through incompetent venous valves, which can cause venous stasis ulcers of the skin and subcutaneous tissue. Venous valve insufficiency can occur, for example, in the superficial venous system, such as the saphenous veins in the leg, or in the deep venous system, such as the femoral and popliteal veins extending along the back of the knee to the groin. Venous insufficiency is caused by compromised vein valves in the leg. Venous insufficiency is recognized in two forms: (1) chronic venous ulcer, and (2) varicose veins. In the United States, chronic venous insufficiency (CVI) associated with skin changes and ulcers effect six to seven million people.

Skin changes and ulcers due to venous insufficiency usually result from valve damage or deep venous occlusion following a bout of DVT. Active venous ulcers are the leading cause of leg ulceration and the long-term healing prognosis, when compared to arterial and diabetic ulcers, is poor. While estimated to be 10 times more common, chronic venous insufficiency has received less attention than arterial insufficiency. CVI is the seventh most debilitating disease in the United States. Principle risk factors associated with venous ulcers include increased age, obesity, male gender, lower extremity trauma, and a history of deep vein thrombosis (DVT).

Varicose veins, the second manifestation of chronic venous insufficiency, occur when walls of the vein lose their elasticity, causing vessel dilation that stretches the valves to incompetence. Varicose veins are estimated to affect 4.2% of the adult western population. It is also estimated that 27% of the United States adult population have some form of detectable lower extremity venous abnormality, primarily varicose veins and telangiectasia. Approximately half of this population has significant varicose veins for which treatment will be sought. Primary risk factors are a history of phlebitis, female gender, and a family history of varicose veins. The varicose vein condition consists of dilatation and tortuousity of the superficial veins of the lower limb and resulting cosmetic impairment, pain and ulceration. Primary varicose veins are the result of primary incompetence of the venous valves of the superficial venous system. Secondary varicose veins occur as the result of deep venous hypertension which has damaged the valves of the perforating veins, as well as the deep venous valves. The initial defect in primary varicose veins often involves localized incompetence of a venous valve thus allowing reflux of blood from the deep venous system to the superficial venous system. This incompetence is traditionally thought to arise at the saphenofemoral junction but may also start at the perforators. Thus, gross saphenofemoral valvular dysfunction may be present in even mild varicose veins with competent distal veins. Even in the presence of incompetent perforation, occlusion of the saphenofemoral junction usually normalizes venous pressure.

The initial defect in secondary varicose veins is often incompetence of a venous valve secondary to hypertension in the deep venous system. Since this increased pressure is manifested in the deep and perforating veins, correction of one site of incompetence could clearly be insufficient as other sites of incompetence will be prone to develop. However, repair of the deep vein valves would correct the deep venous hypertension and could potentially correct the secondary valve failure. A part from the initial defect, the pathophysiology is similar to that of varicose veins.

The methods of treatment preferably include the step of delivering a means for modifying fluid flow to a body vessel, which is preferably a vein or other blood vessel in communication with the venous system. The means for modifying fluid flow may be a flow-modifying medical device that reduces fluid flow in a flow direction-dependent manner. The diameter of the orifice may be substantially invariant to changes in fluid flow direction from the antegrade direction to the retrograde direction. The flow-modifying medical device can be configured to provide a fluid flow restriction channel that comprises an orifice that is substantially constant during changes in fluid flow pressure and/or direction. Preferably, the diameter of the orifice does not substantially change or close to prevent fluid flow in either antegrade or retrograde directions. Rather, fluid flow is desirably reduced in one or both directions, rather than eliminated in either direction.

A method of treating a venous valve related condition can include the step of implanting a flow-modifying medical device within a body vessel having a body vessel diameter. The flow-modifying medical device may include a flow orifice having a diameter that is less than the body vessel diameter, and may be configured to reduce the rate of fluid flow in an antegrade direction across the flow-modifying medical device by less than the reduction of fluid flow in the retrograde direction across the flow-modifying device. The flow-modifying medical device can be implanted at any suitable site in the vasculature. For treatment of venous disease, the medical device is desirably implanted in the superficial venous system, such as the saphenous veins in the leg, or in the deep venous system, such as the femoral and popliteal veins extending along the back of the knee to the groin.

Preferably, the flow-modifying device is implanted percutaneously to a point of treatment in a body vessel using any suitable delivery device, including delivery catheters dilators, sheaths, and/or other suitable endoluminal devices. Alternatively, the flow-modifying device can be placed in body vessels or other desired areas by any suitable technique, including percutaneous delivery as well as surgical placement. According to one embodiment, a flow-modifying medical device may be implanted superficial to the saphenous vein upstream (that is below or away from the heart) of the saphenous junction. Preferably the flow-modifying medical device is placed within about 10 cm or more preferably 5 cm from the junction. In this manner, implantation of the flow-modifying medical device may reduce backflow of the blood to a greater extent than the flow of blood toward the heart due to peripheral venous pumping.

Multiple flow modifying devices can be inserted upstream (with respect to blood flow) and/or downstream of one or more venous valve leaflets. When a venous valve related condition is manifested by failure of valves within both the upper (e.g. saphenous) and lower (e.g. popliteal) portions of the leg, the flow-modifying medical device may also be implanted within a blood vessel lower on the leg alone or more preferably in combination with one or more flow-modifying medical devices implanted higher on the leg such as in the vicinity of the saphenous junction. One particularly suitable location for placement of the flow-modifying medical device is superficial to the saphenous vein immediately upstream of the popliteal junction, for example within about 5 cm from the junction.

Flow-Modifying Devices

In a first embodiment, methods of treating a venous-valve related condition are provided wherein a means for modifying fluid flow within the venous system is implanted within a body vessel. The means for modifying fluid flow preferably includes one or more implantable flow-modifying medical devices as described herein. The flow-modifying medical devices are preferably configured to restrict the rate of fluid flow within a body vessel. The flow-modifying medical device can have a variety of suitable configurations. Most simply, the flow-modifying medical device is an annular ring or tube defining a fluid flow restricting channel adapted to conduct fluid from an inlet to an outlet. The geometry of the fluid flow restricting channel may be selected to provide a desired reduction in the rate of fluid flow across the medical device may be reduced in one or both directions by about 0.5-30% when passing through the flow-modifying medical device, including a reduction of 1, 5, 10, 15, 20, 25 or 30% in the rate of fluid flow across the medical device in the antegrade direction and/or the retrograde direction. Desirably, flow-modifying devices reduce the rate of fluid flow in at least one direction by up to about 25%, including a reduction in flow rate of about 1-25%, including a reduction in flow rate of up to about 1%, 5%, 10%, 15%, 20%, or any percentage therebetween. For example, the flow-modifying device may reduce blood flow within a vein in the antegrade and/or retrograde direction by a total of about 1%, 5%, 10%, 15%, 20%, 25% or more, including a reduction in flow rate of about 1-25%, 1-20%, 1-10%, 1-5%, 10-25%, 15-25% and 10-20%. The channel can have a diameter that is less than the diameter of the inlet and/or outlet of the medical device, thereby reducing the rate of fluid flow within the body vessel. Alternatively, the medical device may define a two or more fluid channels including a first branch defining a fluid flow channel with a diameter that is the same or different from that of an adjoined second branch. For example, a medical device may have a bifurcated “Y-shaped” configuration, permitting fluid in a body vessel to be diverted into one direction at a more rapid rate than in a second direction.

Preferably, the flow-modifying medical device includes a flow restricting channel configured to reduce the rate of fluid flow through a body vessel in a direction-dependent manner, preferably by reducing the rate of fluid flow in a first direction less than in a second direction. The first direction and the second direction may be longitudinally opposite directions, such as antegrade and retrograde fluid flow, or may be disposed at angles of less than 180 degrees with respect to each other, such as a bifurcation of fluid flow in a “Y-shaped” flow channel. Directionally-dependent flow reducing medical devices are exemplified herein by discussion of a longitudinal flow channel conducting fluid in the antegrade or retrograde direction. However, other aspects of the preferred embodiments also pertain to fluid flow channels with alternative flow configurations. Directionally-dependent flow reducing medical devices may reduce the rate of fluid flow more in either the antegrade or retrograde flow direction, but preferably reduce the rate of fluid flow in the retrograde flow direction more than in the antegrade flow direction. Accordingly, the rate of fluid flow in the antegrade direction may be greater than in the retrograde direction. The reduction of fluid flow in the antegrade or retrograde directions are preferably up to about 30%, including a reduction in flow rates of about 1-30% in one or both directions. The reduction in the rate of fluid flow across the medical device in the retrograde direction may be about 0.5-25%, preferably 1-20%, most preferably about 5-20%, including 1, 2, 3, 4, 5, or 6% greater than the rate of fluid flow reduction in the antegrade direction. Within a blood vessel, the flow-modifying device may be adapted to resist blood flow in an antegrade direction toward the heart less than blood flow in a retrograde direction toward veins or blood vessels in fluid communication with veins so as to mitigate symptoms associated with venous valve incompetence (e.g., to reduce pooling of blood in the legs by implanting a flow modifying device in a vein).

By varying the relative cross-sectional areas of the inlet, outlet and orifice, as well as the geometries of the antegrade fluid receiving surface and the retrograde fluid receiving surface, the rate of fluid flow through the fluid flow channel of the flow-modifying device can vary depending on the direction of the fluid flow. For example, two flow-modifying devices described in Example 1 each reduced the rate of fluid flow in an antegrade direction by up to about 23%, but reduced the rate of fluid flow in a retrograde direction by up to about 27%. The reduction of fluid flow was dependent on the pressure head, as indicated in the Examples, however the percentage reduction in fluid flow in the retrograde direction was greater than the percentage reduction in fluid flow in the antegrade direction by between about 1% and 6% at all pressure heads tested, including 1.7%, 1.8%, 2.4%, 2.5%, 3.3%, 4.8%, and 5.4%. Other aspects provide flow-modifying medical devices configured to reduce the rate of fluid flow in the retrograde direction by about 10-20%, preferably about 15-20%, more than the reduction in fluid flow rate in the antegrade direction. Alternatively, the flow-modifying medical device may be configured to reduce the rate of fluid flow across the medical device in the antegrade direction more than fluid flow in the retrograde direction, including a reduction of between about 0.5% and 20% more flow rate reduction in the antegrade direction than in the retrograde direction. Other aspects provide flow-modifying medical devices configured to reduce the rate of fluid flow in the antegrade direction by about 10-20%, preferably about 15-20%, more than the reduction in fluid flow rate in the retrograde direction.

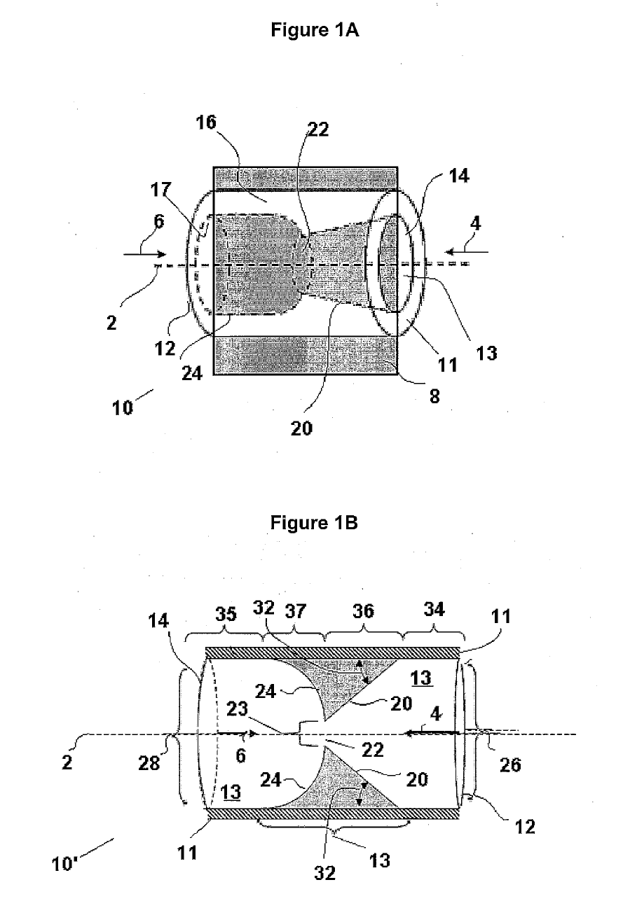

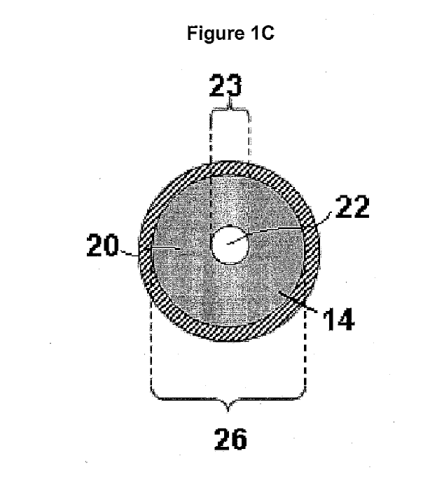

Referring to FIG. 1A, a first flow-modifying medical device 10 comprises a member 11 having a tubular exterior surface 16 and an interior surface 17 defining a bi-directional fluid flow restricting channel 13 extending longitudinally through the member 11 from an inlet 14 to an outlet 12, permitting fluid to flow through an orifice 22 in an antegrade direction 4 or a retrograde direction 6. The interior surface 17 comprises an antegrade fluid receiving surface 20 joined to a retrograde fluid receiving surface 24 at the orifice 22. A longitudinal axis 2 of the member 11 extends through the center of the orifice 22. A hypothetical plane 8 containing the longitudinal axis 2 is shown bisecting the member 11.

FIG. 1B is a cross-sectional view 10′ of the first flow-modifying medical device 10 along the hypothetical plane 8 shown in FIG. 1A. The walls of the tubular member 11 define a bi-directional fluid flow restricting channel 13 that is preferably substantially radially symmetric about a longitudinal axis 2. The antegrade fluid receiving surface 20 is positioned to direct longitudinal fluid flow in an antegrade direction 4 into the orifice 22. The longitudinal thickness of the orifice 22 is preferably minimized, with thicknesses of less than 0.1-inch (2.54 mm) being preferred, such as a thickness of about 0.02-inch (0.508 mm). The retrograde fluid receiving surface 24 is preferably configured to provide a greater resistance to fluid flow in the retrograde direction 6 than the resistance of the antegrade fluid receiving surface 24 provides to fluid flow in the antegrade direction 4.

The antegrade fluid receiving surface 20 may include an angled portion 36 extending from the orifice 22 in the retrograde direction 6. The antegrade fluid receiving surface 20 optionally includes a first annular portion 34 extending longitudinally away from both the orifice 22 and the angled portion 36 in the retrograde direction 6. The first annular portion 34 may have a substantially constant diameter 26 and define the inlet 14, in fluid flow communication with the angled portion 36. The longitudinal length of the annular portion 34 may be, for example, between about 0.1-inch (2.54 mm) and 0.5-inch (12.7 mm), including a length of about 0.36-inch (9.14 mm). Alternatively, first annular portion 34 can be omitted, and the inlet can be defined by the portion of the angled portion 36 distal to the orifice 22. Preferably, the angled portion 36 has a frustoconical cross-sectional geometry. The angled portion 36 of the antegrade fluid receiving surface 20 can be oriented at an angle 32 with respect to the interior surface 17 of the tubular member 11 proximal or distal to the angled portion 36. Typically, an angle congruent to the angle 32 is formed between the angled portion 36 and the longitudinal axis 2. The angle 32 is preferably selected to provide a desired resistance to fluid flow through the channel 13 in the antegrade direction 4. Increasing the angle 32 may increase the resistance to fluid flow in the antegrade direction. The angle 32 is preferably about 20-60 degrees and most preferably about 40-degrees.

The retrograde fluid receiving surface 24 is configured to direct longitudinal fluid flow in the retrograde direction 6 into and through the orifice 22. The retrograde fluid receiving surface 24 extends from the orifice 22 to the outlet 12, which has a diameter 28. At least a portion of the cross-sectional geometry of the retrograde fluid receiving surface 24 may have an arcuate geometry. For example, the retrograde receiving surface 24 can have a curved portion 37 extending from the orifice 22 in the antegrade direction 4, facing opposite the antegrade flow receiving surface 20. The curved portion 37 may have a “bowl”-like geometry, extending away from an orifice 22 located in the bottom of the “bowl.” A transverse cross-section of the “bowl”-like geometry preferably includes a semi-circular arcuate line having radius of curvature of between about 0.1-inch (2.54 mm) and 0.2-inch (5.08 mm), such as a radius of 0.118-inch (about 3 mm) or 0.185-inch (about 4.7 mm). Increasing the radius of curvature of the curved portion 37 may increase the resistance to fluid flowing through the channel 13 in the retrograde direction 6.

The retrograde fluid receiving surface 24 optionally further includes a second annular portion 35 extending longitudinally away from both the orifice 22 and the arcuate in the retrograde direction 6. The second annular portion 35 may have a substantially constant diameter 28 and define the outlet 12, in fluid flow communication with a curved portion 35. The longitudinal length of the second annular portion 35 may be the same or similar to the longitudinal length of the first annular portion 34.

Preferably, the total length measured along the longitudinal axis 2 of the curved portion 37, the orifice 22 and the angled portion 36 is preferably about 0.1-inch (2.54 mm) to about 0.25-inch (6.35 mm), including lengths of about 0.217-inch (about 5.5 mm) and 0.327-inch (about 8.3 mm). The diameter 28 is preferably substantially equal to the diameter 26, preferably about 0.25-inch (about 6.4 mm) to 0.75-inch (about 19 mm), and most preferably about 0.40-inch (about 10.2 mm) and 0.60-inch (about 15.3 mm), including diameters of about 0.51-inch (about 13.0 mm) or 0.425-inch (about 10.8 mm).

While the exemplary medical devices described above comprise a fluid flow restricting channel 13 having an asymmetric cross section in the radial plane 8 containing the longitudinal axis 2, alternative fluid flow restricting channel 13 geometries are also provided. For example, the antegrade flow receiving surface 20 may have an arcuate cross section and/or the retrograde flow receiving surface 24 may have a frustoconical cross section. One aspect provides a flow-modifying medical device having antegrade flow receiving surface 20 and a retrograde flow receiving surface 24 both having a frustoconical cross-sectional geometry that are the same or different. Another aspect provides a flow-modifying medical device having antegrade flow receiving surface 20 and a retrograde flow receiving surface 24 both having an arcuate cross-sectional geometry that are the same or different. In addition, the orifice 22 may have any suitable geometry, including circular or elliptical. In yet another aspect, the fluid flow restricting channel 13 is defined by an annular ring, including a ring having a thickness that is less than the diameter of the ring, more preferably a ring having a thickness that is less than 20%, 10%, 5% or 1% of the ring diameter. Alternatively, the fluid flow restricting channel 13 may comprise an antegrade flow receiving surface 20 having an arcuate cross-sectional geometry, and the retrograde flow receiving surface 24 may have a frusto-conical cross-sectional geometry. Optionally, the thickness of the orifice 22 may be extended to form a channel between the antegrade flow receiving surface 20 and the retrograde flow receiving surface 24.

Flow-modifying devices comprising an antegrade or retrograde flow receiving surface with a frustoconical cross-sectional geometry may include a surface at an angle selected to provide a desired resistance to fluid flow in the antegrade or retrograde direction. In generally, increasing the angle may increase the resistance to fluid flow toward the orifice. Desirably, the angle of a frustoconical antegrade or retrograde fluid flow receiving surface is about 20-70, 30-60, 35-45, or about 40-45 degrees, including any suitable angle therebetween. The frustoconical cross-sectional shape desirably comprises a radially symmetric surface angled around the fluid flow restricting channel 13. However, alternative embodiments provide frustoconical antegrade or retrograde flow receiving surfaces having longitudinal cross-sectional geometries comprising a first subtended angle and a second subtended angle with respect to the other flow receiving surface.

FIG. 1C is an end view of the inlet 14 of the first flow-modifying device, showing the antegrade flow receiving surface 20 extending from the orifice 22, having a diameter 23, to the inlet 14, having a diameter 26. The orifice 22 preferably has a cross-sectional area (i.e., π×((diameter 23)/2)²) that is less than the first cross-sectional area (i.e., π×((diameter 26)/2)²) of the inlet and/or a second cross-sectional area of the outlet (not shown). The orifice 22 can have any suitable configuration, but has a substantially circular shape with a diameter of about 3, 4, 5, 6, 7, 8 mm or more. Preferably, the diameter 23 of the orifice is about 5.5-6.0 mm (e.g., 0.217-inch or 0.238-inch) and the diameter 26 of the inlet 14 is about 11-14 mm. The ratio between the cross-sectional area of the inlet and the cross-sectional area of the orifice is desirably between about 1.0 and 5.0, including ratios of 0.1 therebetween, and preferably between about 2.0 and 2.5, including ratios of about 2.0 and 2.2. The cross-sectional area at the outlet is preferably substantially equal to the cross-sectional area at the inlet. The cross-sectional area is measured perpendicular to the longitudinal axis 2. As described above, the total length of (1) the longitudinal length of the curved portion 37 of the retrograde fluid receiving surface 24, (2) the thickness of the orifice 22 and (3) the longitudinal length of the angled portion 36 of the antegrade fluid receiving surface 20 is preferably about 0.1-inch (2.54 mm) to about 0.25-inch (6.35 mm), including lengths of about 0.217-inch (about 5.5 mm) and 0.327-inch (about 8.3 mm). The ratio of this total length to the diameter of the orifice 22 is preferably about 1.00 to 1.50, including a ratio of about 1.37. The ratio between the diameter 28 at the outlet 12 (or the diameter 26 at the inlet 14) and the thickness of the orifice 22 is preferably about 15-25, most preferably about 20-25 (including ratios of 25.5 and 21.25). The diameter 28 at the outlet 12 and the diameter 26 at the inlet 14 are preferably substantially equal.

The fluid contact surface defining the flow restricting channel can be formed from any suitable material. In one aspect, such as medical device 10, a solid tubular member 11 can have an interior surface 17 contiguously defining a fluid flow restricting channel 13. The solid tubular member 11 can be formed from a thermoformable polymer, such as polyethylene, polyurethane, or polypropylene. The tubular member 11 may be biostable or bioabsorbable, and can be configured in any manner suitable for a desired use that provides a desired level of flexibility or rigidity for an intended application. For stenting applications, the tubular member 11 is typically a tubular structure formed from a biostable material having a substantially annular cross-sectional configuration, with an interior surface defining a tubular lumen extending longitudinally through the support member. The tubular member 11 is preferably formed from any suitable material, such as a polyethylene or polyurethane that is thermoformable, biocompatible and provides desired levels of rigidity.

The medical device 10 may optionally be configured as a biostable tubular member 11 enclosing a biodegradable material defining at least a portion of the interior surface 17 defining the antegrade flow receiving surface 20, the orifice 22 or the retrograde flow receiving surface 24. The biodegradable material can be formed from a variety of biodegradable polymers, and may include a biodegradable material selected to provide a desired rate of absorption within the body. The biodegradable material can be chosen to degrade or be absorbed within a body over a period of weeks or months. Certain biodegradable polymers are known to degrade within the body at differing rates based upon the polymer selected and the location of implantation, such as within a body vessel. Poly(lacetic acid), poly(glycolic acid), poly(caprolactone) and copolymers or mixtures thereof are particularly preferred biodegradable materials.

Forming Films

In another aspect, the fluid contact surface is formed from a forming material, such as biocompatible polymer, configured to form the antegrade and retrograde flow receiving surfaces. The biocompatible polymer is selected to be thermoformable at a desired forming temperature while retaining its shape after cooling. For example, the forming film can be shaped by dipping a mandrel into a melted, dissolved or softened thermoformable polymer that adheres to and assumes the shape of the mandrel after drying the polymer onto the mandrel. Optionally, a flow modifying medical device can further comprise a radially expandable frame attached to the forming film.

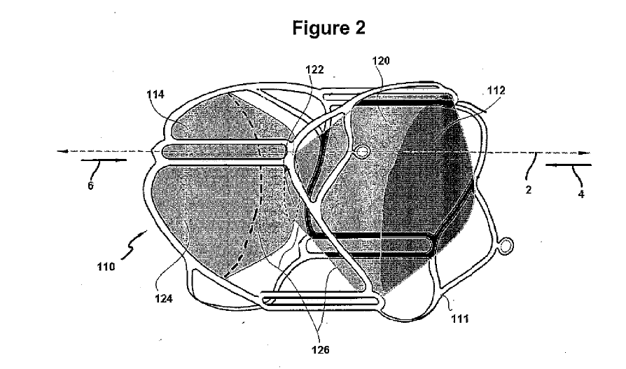

FIG. 2 is a perspective view of a variation of the flow-modifying medical device comprising fluid flow channel formed from a forming film attached to an implantable frame. A second flow-modifying medical device 110 is shown comprising a forming film 126 formed from a biocompatible polyurethane attached to an implantable frame 111 disposed around the forming film 126. The forming film 126 forms a bi-directional fluid flow restricting channel extending along the longitudinal axis 2 from an inlet 112 to an outlet 114. The forming film 126 defines an antegrade flow receiving surface 120 and a retrograde flow receiving surface 124 joined at an orifice 122. The forming material 126 is rigid enough to direct fluid into and through the orifice 122 from either longitudinal direction. By configuring the retrograde flow receiving surface 124 and the antegrade flow receiving surface 120 differently, fluid flow passing though the orifice 122 is preferably reduced more in the retrograde direction 6 than in the antegrade direction 4. The forming film can be attached to a suitable support frame 111 configured to maintain the fluid flow restricting channel in a desired geometry. The support frame can optionally provide a stenting function to the medical device (i.e., exert a radially outward force on the interior wall of a vessel in which the medical device is implanted). By including a support frame that exerts such an outward radial force, a medical device can provide both a stenting and a flow-modifying function at a point of treatment within a body vessel.

The forming film 126 can be formed from any suitable material. Preferably, the forming film 126 is impervious to fluid, although porous materials can be used. The antegrade flow receiving surface 120 can be formed from any material configured to provide enough resistance to fluid flow in the antegrade direction 4 to direct the fluid through the orifice 122. The retrograde flow receiving surface 124 is desirably formed from the same material as the antegrade flow receiving surface 120. Most preferably, the forming film 126 comprises a biocompatible polyurethane.

Optionally, a blood-contacting surface of the forming film may be modified to mitigate thrombus formation. In one aspect, heparin or a heparin derivative may be attached to the surface of a polyurethane forming film. For example, a covalently bonded conjugate of commercial grade heparin and prostaglandin E1 (PGE1) may be immobilized on the surface of the forming film, through hydrophilic spacer groups (diamino-terminated polyethylene oxides). One end of the spacer group may be coupled to the derivatized surface through a urethane bond between the amine group of the spacer and the derivatized surface. The free amine group of the immobilized spacers may be coupled to a carboxylic group of the PGE1-heparin conjugate through an amide bond. See H. A. Jacobs, et al., “Antithrombogenic surfaces: characterization and bioactivity of surface immobilized PGE1-heparin conjugate,” J Biomed Mater Res. 1989 June; 23(6):611-30.

The forming film 126 preferably comprises a biocompatible modified polyetherurethane. One particularly preferred forming film is constructed from a polyetherurethane/polysiliconeurethane, such as the material sold under the tradename THORALON® (THORATEC, Pleasanton, Calif.). THORALON® has been used in certain vascular applications and is characterized by thromboresistance, high tensile strength, low water absorption, low critical surface tension, and good flex life. THORALON® is believed to be biostable and to be useful in vivo in long term blood contacting applications requiring biostability and leak resistance. Because of its flexibility, THORALON® is useful in larger vessels, such as the abdominal aorta, where elasticity and compliance is beneficial.

The forming film 126 is preferably a non-porous polymer comprising polyurethane, although a porous polyurethane materials can also be used, as described below. Implantable medical devices can comprise non-porous and/or porous forms of THORALON. The thromboresistant material preferably comprises the non-porous form of THORALON. As described in U.S. Pat. Application Publication No. 2002/0065552 A1 and U.S. Pat. No. 4,675,361, both of which are incorporated herein by reference, the biocompatible polyurethane material, such as THORALON®, is preferably a polyurethane base polymer (referred to as BPS-215) blended with a siloxane containing surface modifying additive (referred to as SMA-300). The concentration of the surface modifying additive may be in the range of 0.5% to 5% by weight of the base polymer. The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference. The BPS-215 component (THORATEC) is a segmented polyetherurethane urea containing a soft segment and a hard segment. The soft segment is made of polytetramethylene oxide (PTMO), and the hard segment is made from the reaction of 4,4-diphenylmethane diisocyanate (MDI) and ethylene diamine (ED).

Alternatively, the forming film 126 can comprise a porous material, such as a porous form of THORALON. Porous THORALON can be formed by mixing the polyetherurethane urea (BPS-215), the surface modifying additive (SMA-300) and a particulate substance in a solvent. The particulate may be any of a variety of different particulates or pore forming agents, including inorganic salts. Preferably the particulate is insoluble in the solvent. The solvent may include dimethyl formamide (DMF), tetrahydrofuran (THF), dimethyacetamide (DMAC), or dimethyl sulfoxide (DMSO), or mixtures thereof. The composition can contain from about less than 1 wt % to about 40 wt % polymer, and different levels of polymer within the range can be used to fine tune the viscosity needed for a given process. The composition can contain less than 5 wt % polymer for some spray application embodiments, such as 0.1-5.0 wt %. For dipping application methods, compositions desirably comprise about 5 to about 25 wt %. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water for the extraction, for example water at a temperature of about 60° C. The resulting pore diameter can also be substantially equal to the diameter of the salt grains. The resulting void-to-volume ratio, as defined above, can be substantially equal to the ratio of salt volume to the volume of the polymer plus the salt. Formation of porous THORALON is described, for example, in U.S. Pat. Application Publication Nos. 2003/0114917 A1 and 2003/0149471 A1, both of which are incorporated herein by reference.

A variety of other biocompatible polyurethanes/polycarbamates and urea linkages (hereinafter “—C(O)N or CON-type polymers”) may also be employed in the forming film 126. These include CON type polymers that preferably include a soft segment and a hard segment. The segments can be combined as copolymers or as blends. For example, CON type polymers with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole. Preferably, the hard segment is formed from a diisocyanate and diamine. The diisocyanate may be represented by the formula OCN—R—NCO, where —R— may be aliphatic, aromatic, cycloaliphatic or a mixture of aliphatic and aromatic moieties. Examples of diisocyanates include MDI, tetramethylene disocyanate, hexamethylene diisocyanate, trimethyhexamethylene diisocyanate, tetramethylxylyiene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-d iisocyanate, 1-methoxyphenyl 2,4-d iisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures. Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8-octanediol, propylene glycols, 2,3-butylene glycol, dipropylene glycol, dibutylene glycol, glycerol, or mixtures thereof. Biocompatible CON type polymers modified with cationic, anionic and aliphatic side chains may also be used. See, for example, U.S. Pat. No. 5,017,664. Other biocompatible CON type polymers include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif.). Other biocompatible CON type polymers can include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference. Optionally, any of these biocompatible CON type polymers may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

Support Frames

The medical device 110 can also comprise a means for supporting the forming film to form a fluid flow restricting channel, such as the support frame 111, for providing structural support to the forming film (e.g., to maintain the forming film in the geometry desired to form the fluid flow restricting channel). Typically, a support frame 111 is attached to the forming film 126. The support means can be formed from any suitable structure that maintains an attached thromboresistant material in a desired position, orientation or range of motion to perform a desired function. Preferably, the support means is a radially expandable support frame adapted for implantation within a body vessel from a delivery catheter.

The implantable frame 111 preferably defines a fluid flow restricting channel having substantially cylindrical or elliptical cross-sectional geometry. The frame structure may comprise a plurality of struts, which can be of any suitable structure or orientation. In some embodiments, the frame comprises a plurality of struts connected by alternating bends. For example, the frame can be a ring or annular tube member comprising a series of struts in a “zig-zag” pattern. The frame can also comprise multiple ring members with struts in a “zig-zag” pattern, for example by connecting the ring members end to end, or in an overlapping fashion. In some embodiments, the struts are substantially aligned along the surface of a tubular plane, and substantially parallel to the longitudinal axis of the support frame. Support frames can also be formed from braided strands of one or more materials, helically wound strands, ring members, consecutively attached ring members, tube members, and frames cut from solid tubes. The support frame 111 can have any suitable configuration and size. The support frame can be sized so that the second, expanded configuration is slightly larger in diameter that the inner diameter of the vessel in which the medical device will be implanted. This sizing can facilitate anchoring of the medical device within the body vessel and maintenance of the medical device at a point of treatment following implantation. Preferably, the support frame is configured for implantation in a body vessel having an inner diameter of about 5 mm to about 25 mm, more preferably about 8 mm to about 15 mm.

The implantable frame 111 may be formed from any suitable biocompatible material that allows for desired therapeutic effects upon implantation in a body vessel. Examples of suitable materials include, without limitation, any suitable metal or metal alloy, such as: stainless steels (e.g., 316, 316L or 304), nickel-titanium alloys including shape memory or superelastic types (e.g., nitinol or elastinite); inconel; noble metals including copper, silver, gold, platinum, palladium and iridium; refractory metals including molybdenum, tungsten, titanium, rhenium, or niobium; stainless steels alloyed with noble and/or refractory metals; magnesium; amorphous metals; plastically deformable metals (e.g., tantalum); nickel-based alloys (e.g., including platinum, gold and/or tantalum alloys); iron-based alloys (e.g., including platinum, gold and/or tantalum alloys); cobalt-based alloys (e.g., including platinum, gold and/or tantalum alloys); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; platinum-iridium alloys; platinum-tungsten alloys; magnesium alloys; titanium alloys (e.g., TiC, TiN); tantalum alloys (e.g., TaC, TaN); L605; bioabsorbable materials; or other biocompatible metals and/or alloys thereof. Preferably, the implantable frame comprises a self-expanding nickel titanium (NiTi) alloy material, stainless steel or a cobalt-chromium alloy.

Optionally, an implantable frame 111 can comprise a core layer of a metal base material coated with a bioabsorbable material, such that absorption of the bioabsorbable material changes the flexibility of the frame after a desirable period of implantation in a body vessel. In some embodiments, a frame comprises a biostable core or “base” material surrounded by, or combined, layered, or alloyed with a bioabsorbable material. Preferably, a bioabsorbable, biocompatible polymer is approved for use by the U.S. Food and Drug Administration (FDA). These FDA-approved materials include polyglycolic acid (PGA), polylacetic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). In general, these materials biodegrade in vivo in a matter of months, although some more crystalline forms can biodegrade more slowly.

Preferably, the frame material is a self-expanding material capable of significant recoverable strain to assume a low profile for delivery to a desired location within a body lumen. After release of the compressed self-expanding resilient material, it is preferred that the frame be capable of radially expanding back to its original diameter or close to its original diameter. Accordingly, some embodiments provide frames made from material with a low yield stress (to make the frame deformable at manageable balloon pressures), high elastic modulus (for minimal recoil), and is work hardened through expansion for high strength. Particularly preferred materials for self-expanding implantable frames are shape memory alloys that exhibit superelastic behavior, i.e., are capable of significant distortion without plastic deformation. Frames manufactured of such materials may be significantly compressed without permanent plastic deformation, i.e., they are compressed such that the maximum strain level in the resilient material is below the recoverable strain limit of the material. Discussions relating to nickel titanium alloys and other alloys that exhibit behaviors suitable for frames can be found in, e.g., U.S. Pat. No. 5,597,378 (Jervis) and WO 95/31945 (Burmeister et al.). A preferred shape memory alloy is Ni—Ti, although any of the other known shape memory alloys may be used as well. Such other alloys include: Au—Cd, Cu—Zn, In—Ti, Cu—Zn—Al, Ti—Nb, Au—Cu—Zn, Cu—Zn—Sn, CuZn—Si, Cu—Al—Ni, Ag—Cd, Cu—Sn, Cu—Zn—Ga, Ni—Al, Fe—Pt, U—Nb, Ti—Pd—Ni, Fe—Mn—Si, and the like. These alloys may also be doped with small amounts of other elements for various property modifications as may be desired and as is known in the art. Nickel titanium alloys suitable for use in manufacturing implantable frames can be obtained from, e.g., Memory Corp., Brookfield, Conn. One suitable material possessing desirable characteristics for self-expansion is Nitinol, a Nickel-Titanium alloy that can recover elastic deformations of up to 10 percent. This unusually large elastic range is commonly known as superelasticity.

Alternative Flow-Modifying Device Configurations

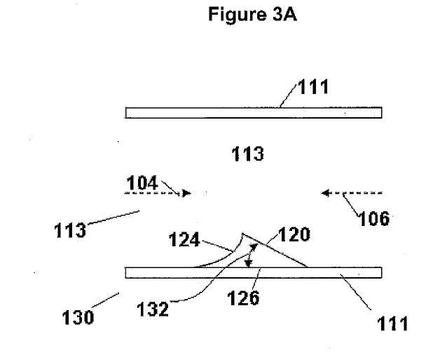

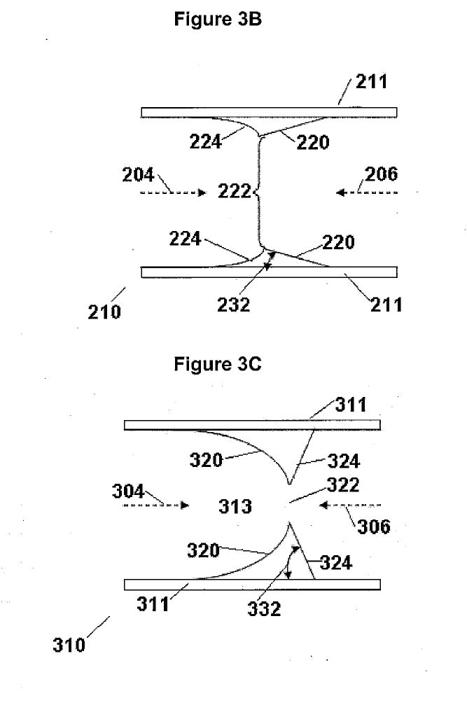

Optionally, the flow-modifying medical device may comprise a radially asymmetric flow restricting channel, such as a flow receiving surface positioned on one side of a tubular channel. For example, FIG. 3A shows a cross-sectional view of a third flow-modifying medical device 130 having an antegrade flow receiving surface 120 at an angle 132, mounted to an outer tube member 111. Fluid is permitted to flow longitudinally through the medical device 130 in an antegrade direction 106 and a retrograde direction 104. The medical device 130 comprises a radially asymmetric flow-modifying surface on one side of the flow restricting channel 113.

Alternatively, the flow-modifying device 130 may be modified by omitting the tube member 111, and implanting a medical device having a three-sided structure comprising the antegrade flow receiving surface 120, the retrograde flow receiving surface 124 and the wall engaging surface 126. The wall-engaging surface 126 can be adapted for implantation within a body vessel by attachment to the wall of the body vessel, for example by forming a plurality of barbs in the wall-engaging surface 126 or by including a suitable adhesive material on the wall-engaging surface 126.

In another aspect, the flow-modifying medical device may have an antegrade flow receiving surface oriented at different angles with respect to the retrograde flow receiving surface. Referring to FIG. 3B and FIG. 3C, two substantially similar flow-modifying devices differ only with respect to the angle subtended by the antegrade flow receiving surface. FIG. 3B shows a cross-sectional view of a fourth flow-modifying device 210 comprising an outer tube 211 defining a fluid flow restricting channel 213 having a frustoconical antegrade flow receiving surface 220 at a first angle 232 within the fluid flow restricting channel 213 comprising an orifice 222. Fluid flow in the retrograde direction 204 is reduced to a greater extent upon passing through the orifice 222 than fluid flow in the antegrade direction 206. FIG. 3C shows a cross-sectional view of a modified fourth flow-modifying device 310 comprising an outer tube 311 defining a fluid flow restricting channel 313 having a frustoconical antegrade flow receiving surface 324 at a second angle 332 (that is greater than the first angle 232 in FIG. 3B) with respect to an arcuate retrograde flow receiving surface 320 within the fluid flow restricting channel 313 comprising an orifice 322. Fluid flow in the retrograde direction 304 is reduced to a greater extent upon passing through the orifice 322 than fluid flow in the antegrade direction 306.

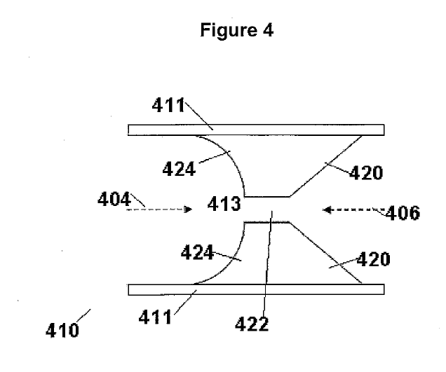

In another aspect, the fluid flow restricting channel can optionally comprise a channel between the antegrade receiving surface and the retrograde receiving surface. Referring to FIG. 4, a cross section of a fifth flow-modifying medical device 410 shows an external tube member 411 defining a flow restricting channel 413, and an antegrade flow receiving surface 420 and a retrograde flow receiving surface 424 in communication with opposite ends of an orifice channel 422 therebetween. Optionally, the fifth flow-modifying medical device 410 may be modified by omitting the external tube member 411 and implanting a structure defining all or part of the antegrade flow receiving surface and the retrograde flow receiving surface.

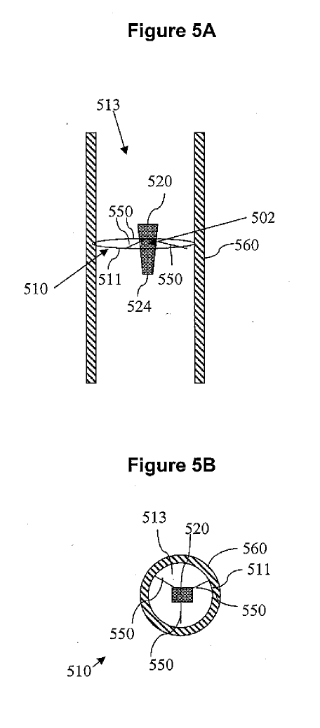

Other aspects of the first embodiment provide flow modifying medical devices such as the sixth medical device 510 shown in FIG. 5A and FIG. 5B. FIG. 5A is a longitudinal side view showing the medical device 510 implanted within a section of a body vessel 560. The body vessel 560 is desirably a vein. The medical device 510 comprises a flow modifying flow diversion means 502 secured within the lumen 513 of the body vessel 560 by a plurality of attachment members 550 attached to a ring 511. The ring 511 is secured to the wall of the body vessel 560 by any suitable means, for example by self-expansion or by including barbs or an adhesive on a portion of the exterior surface of the ring. The flow diversion means 502 can be any structure configured to divert fluid flow around the structure while passing through the lumen 513 of the body vessel 560. Other structures for the flow diversion means 502 include a cylinder, sphere, or geometric block structure mounted at any position within the lumen 513 by one or more attachment members 550. The flow diversion means 502 of the medical device 510 can be any suitable structure that functions to desirably divert fluid flow within the lumen 513, but the structure preferably includes an antegrade flow receiving surface 520 and a retrograde flow receiving surface 524, and diverts fluid flow in an annular fashion around the flow diversion means 502. FIG. 5B shows a top view of the medical device 510 showing the antegrade flow receiving structure 520 attached to the ring 560 by a plurality of attachment members 550. The ring 560 is positioned within the lumen 513 and attached to the wall of the body vessel 560.

Delivery of Flow Modifying Medical Devices

Methods of treating a subject, which can be animal or human, may comprise the step of implanting one or more flow-modifying medical devices as described herein. Methods of treatment may include different delivery approaches. In one aspect, a method of treatment may include the steps of inserting the flow-modifying medical device in an antegrade fashion intravascularly in the popliteal vein. A delivery catheter containing the flow-modifying device may be inserted over a conventional guidewire to permit the distal tip of the catheter shaft to extend beyond (i.e. downstream of) a venous valve leaflet extending annularly from vessel wall. In another aspect, one or more flow-modifying medical devices may be implanted by retrograde insertion of the vascular device. In this approach the delivery catheter is inserted in a direction against the blood flow such that the distal tip of the delivery catheter may extend past a set of valve leaflets in the popliteal vein. The delivery catheter may be inserted through the right jugular vein, where it may be advanced through the superior and inferior vena cava, past the iliac vein, through the femoral vein and into the popliteal vein through leaflets in a retrograde fashion, i.e. opposite the direction of blood flow. The delivery catheter may be placed in the right femoral vein, where it may be advanced in a retrograde manner to the popliteal vein. In a contralateral approach, the delivery catheter may be inserted through the left femoral vein, where it may be advanced around the iliac vein and through the left femoral vein into the popliteal vein. The catheter may be positioned so the flow-modifying medical device may be deployed upstream of the leaflets. The deployment of the flow modifying medical device is otherwise the same as in antegrade insertion procedure above. In yet another aspect, when the flow modifying medical device may be placed downstream (with respect to the direction of blood flow) of a set of venous valve leaflets, the delivery catheter may be inserted in the same antegrade manner as described above, except by being advanced sufficiently past the valve leaflets to enable downstream delivery of the device. The position of the flow-modifying medical device may be confirmed by venography, intravascular ultrasound, or other means and the flow-modifying medical device may be deployed within the body vessel.

A delivery catheter may be placed into the popliteal vein in a patient's leg and advanced to a region adjacent one or more venous valve leaflets to deploy one or more flow-modifying medical devices upstream of the leaflets. The delivery catheter may be thus delivered in an antegrade fashion, with the tip extending downstream of leaflets to deploy the device just upstream (defined in reference to the direction of blood flow) of the leaflets. Alternatively one or more flow modifying medical devices can be inserted to a position downstream of the valve leaflets to deliver the vascular device downstream of the valve leaflets. A delivery device can be inserted through the jugular vein or femoral vein into the popliteal vein or the saphenous vein, or directly into these veins. Optionally, one flow-modifying medical device may be implanted in the saphenous or popliteal vein and one or more additional flow-modifying medical devices may be implanted within a body vessel in communication therewith, including placement of a second flow modifying medical device within the same vein.

There are several different methods of insertion of the flow-modifying medical device, for example to treat venous valve insufficiency of the popliteal or saphenous vein. A delivery catheter adapted to deploy a flow-modifying medical device from a distal portion of the catheter may be placed into the popliteal vein in the patient's leg and advanced to a region adjacent or proximate to a pair of venous valve leaflets to deploy the flow-modifying medical device upstream of the valve leaflets. The delivery catheter may be delivered in an antegrade fashion, with the tip extending downstream of leaflets to deploy the device just upstream (defined in reference to the direction of blood flow) of the leaflets. The delivery catheter may be inserted through the right jugular vein, where it may be advanced through the superior and inferior vena cava, past the iliac vein, through the femoral vein and into the popliteal vein through leaflets in a retrograde fashion, i.e. opposite the direction of blood flow. The delivery catheter may thus extend through the leaflet region just upstream of the leaflets. The delivery catheter may be placed in the right femoral vein, where it may be advanced in a retrograde manner to the popliteal vein in the manner described above. In a contralateral approach, a delivery catheter may be inserted through the left femoral vein, where it may be advanced around the iliac vein and through the left femoral vein into the popliteal vein.

These methods of treatment preferably comprise the step of implanting one or more flow-modifying devices described herein. Preferably, the medical devices described herein are implanted from a portion of a catheter inserted in a body vessel. The flow-modifying medical device may be compressed around the distal portion of a delivery catheter. One method of deploying the flow-modifying device in a vessel involves radially compressing and loading the frame into a delivery device, such as a catheter. A restraining means may maintain the flow-modifying device in the radially compressed configuration. For example, a self-expanding flow-modifying device may be retained within a slidable sheath, while flow-modifying devices that are not self-expanding may be crimped over a balloon portion of a delivery catheter. The compressed flow-modifying device is thereby mounted on the distal tip of the delivery device, translated through a body vessel on the delivery device, and deployed from the distal end of the delivery device. For example, a delivery device may be a catheter comprising a pushing member adapted to urge the flow-modifying device away from the delivery catheter. A sheath may be longitudinally translated relative to the flow-modifying device to permit the flow-modifying device to radially self-expand at the point of treatment within a body vessel. Alternatively, a balloon may be inflated to radially expand the flow-modifying device.

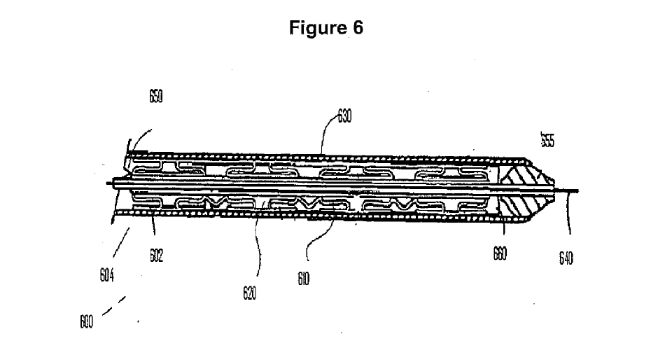

FIG. 6 shows a flow modifying medical device 602 in a radially compressed configuration 600 within the distal end of a delivery catheter 650. The flow modifying medical device 602 is preferably configured as a device described herein. The flow-modifying device 602 may be moveable from the radially compressed configuration to a radially expanded configuration during implantation at a point of treatment within a body vessel. The delivery catheter 650 may include one or more flow-modifying medical devices 602 in a radially compressed configuration while having a low profile and desired flexibility to navigate small or tortuous paths through a variety of body vessels. The flow modifying medical device 602 may be radially expandable from a low profile, radially compressed configuration to an expanded configuration by balloon expansion or self-expansion. The delivery catheter 650 may optionally include a sheath covering and restraining a self-expanding flow-modifying medical device 602 prior to delivery.

The embodiment illustrated in FIG. 6 shows a balloon expandable flow modifying medical device 602. The delivery catheter 650 includes a balloon 660 that is annularly enclosed by the medical device 602 and is inflatable to radially expand the flow modifying medical device 602 by outward pressure. Alternatively, the flow modifying medical device 602 can include a self-expanding ring member and the delivery catheter 650 can be provided without the balloon. Instead, a delivery catheter 650 for a self-expanding medical device may include a retractable means for restraining the medical device in a radially compressed state. The means for restraining the medical device can be retracted to permit the medical device to expand to a radially expanded configuration. Typically, a self-expanding medical device can have a smaller outer diameter in the radially compressed state than a balloon expandable medical device. Delivery catheters 650 for balloon expandable medical devices typically have an outer diameter of about 12-15 french, while the diameter of delivery catheters for self-expanding medical devices are typically about 10-12 french. The medical device 602 extends from the proximal end 604 to the distal end 660 of the catheter 650.

In operation, the delivery catheter 650 may be fitted over a wire guide 640 for delivery to a blood vessel such as an artery or vein by conventional percutaneous transluminal methods. The distal portion 655 of the delivery catheter 650 can be placed within a body vessel at a desired point of treatment, and the balloon 660 can be inflated. The medical device 602 may be deployed by radial expansion of the balloon 660 within a body vessel. The catheter 650 is positioned at a point of treatment within a body vessel. The balloon 660 is then inflated to expand the medical device 602 to the radially expanded configuration. Upon inflation of the balloon, the covering material 630 contacts the interior surface of the body vessel. Subsequently, the balloon 660 can be deflated and the delivery catheter 650 removed from the body vessel along the wire guide 640.

Alternatively, the support frame 610 can comprise a self-expanding material such as a self-expanding nickel-titanium alloy (e.g., nitinol). A medical device 602 comprising a self-expanding support frame 610 can be deployed from a catheter that includes a moveable sheath containing the support frame instead of a balloon. The sheath can be longitudinally translated with respect to the medical device, away from the distal end of the delivery catheter. When the sheath no longer covers the medical device, the self-expanding support frame can radially expand to contact the inner wall of the body vessel, where the medical device can be maintained by the outward force exerted by the frame or by barbs or perforations in the exterior surface of the medical device. The delivery catheter 650 for delivery of a self-expanding medical device may be positioned in a body vessel and the retractable means for restraining the medical device can be retracted to permit radial expansion of the medical device until the covering material 630 contacts the wall of the body vessel.

The delivery catheter 650 is one example of a suitable catheter-based delivery system. Other examples of delivery catheters include the delivery systems disclosed in US patent applications, all of which are incorporated herein by reference in their entirety: Ser. No. 11/382,966 filed May 12, 2006 (2006/0259115 A1); Ser. No. 11/376,864 filed Mar. 16, 2006 (2006/0212107 A1); Ser. No. 11/210,998 filed Aug. 24, 2005 (2006/0058865 A1); Ser. No. 11/123,312 filed May 6, 2005 (2005/0283178 A1); and Ser. No. 10/804,386 filed Mar. 19, 2004 (20040225322 A1).

### EXAMPLES

### Example 1

### Measuring Fluid Flow Rate Through a Flow-Modifying Device

Two flow-modifying medical devices having a configuration of the medical device 110 illustrated in FIG. 2, with a polyurethaneurea (THORALON) forming film 126, were constructed and the rate of fluid flow through the flow restricting channel was measured in the antegrade and retrograde directions. The first flow-modifying medical device (Device 1) had an inlet diameter of 11 mm and an orifice diameter of 5.5 mm, while the second flow-modifying medical device (Device 2) had an inlet diameter of 13.2 mm and an orifice diameter of 6 mm. For both devices, the antegrade flow receiving surface was characterized by a frusto-conical cross-sectional geometry in a hypothetical plane including the longitudinal axis. The angled portion of the flow receiving surface was oriented at about 50-degrees with respect to the longitudinal axis. The thickness of the orifice was minimized at not more than about 0.5 mm thick. The retrograde receiving surface had a bowl-shaped geometry in a hypothetical plane including the longitudinal axis. The radius of the curvature of the curved portion of the retrograde receiving surface was about 4.7 mm in Device 2 and about 3.0 mm in Device 1. The total longitudinal length of the angled portion of the antegrade flow receiving surface, the orifice and the arcuate portion of the retrograde flow receiving surface was about 8.3 mm in Device 2 and about 5.51 mm in Device 1. An annular section of the forming film extended for about 9-10 mm in the antegrade and retrograde directions, each having a diameter substantially equal to the inlet and outlet.

The rate of water flow through the flow restricting channel was measured in each direction as a function of pressure head, and the results are summarized below in Table 1. For each test, fluid flow was measured for about 30 seconds (29.83-30.13 sec). The percent fluid flow rate reduction was calculated as a function of pressure head (measured in mm mercury) compared to a control. Each control was a polyurethaneurea tube with a constant diameter mounted within an annular frame to form each of the flow-modifying devices. The flow rate through each flow-modifying device was then calculated in the antegrade and retrograde directions at each fluid flow pressure. The percentage of flow rate reduction through each flow-modifying device was calculated in comparison with the comparable control. For example, CONTROL-1 was a tube having a diameter of the inlet of flow-modifying device 1. The 1-Antegrade sample refers to the antegrade flow reduction through Device 1 compared to the flow through the CONTROL-1 tube.

TABLE 1

| | | | | % Flow |
|---|---|---|---|---|
| | Pressure | | Standard Dev. | Reduction |
| (Device-Test | Head | Flow Rate | Of Flow Rate | (compared to |
| Description) | (mm Hg) | (mL/min) | (mL/min) | Control) |
| CONTROL-1 | 2 | 1934 | 34.50 | — |
| 1-Antegrade | 2 | 1555 | 8.52 | 20 |
| 1-Retrograde | 2 | 1523 | 3.98 | 21 |
| CONTROL-2 | 2 | 1943 | 1.98 | — |
| 2-Antegrade | 2 | 1704 | 4.59 | 12 |
| 2-Retrograde | 2 | 1655 | 30.80 | 15 |
| CONTROL-1 | 10 | 2489 | 4.56 | — |
| 1-Angegrade | 10 | 1963 | 6.25 | 21 |
| 1-Retrograde | 10 | 1921 | 3.14 | 23 |
| CONTROL-2 | 10 | 2488 | 1.21 | — |
| 2-Antegrade | 10 | 2133 | 1.5 | 14 |
| 2-Retrograde | 10 | 2088 | 0.97 | 16 |
| CONTROL-1 | 50 | 4202 | 91.29 | — |
| 1-Antegrade | 50 | 3320 | 11.78 | 21 |
| 1-Retrograde | 50 | 3118 | 61.26 | 26 |
| CONTROL-2 | 50 | 4177 | 3.51 | — |
| 2-Antegrade | 50 | 3584 | 62.81 | 14 |
| 2-Retrograde | 50 | 3485 | 8.91 | 17 |
| CONTROL-1 | 80 | 5176 | 27.55 | — |
| 1-Antegrade | 80 | 4000 | 9.29 | 23 |
| 1-Retrograde | 80 | 3830 | 8.78 | 26 |
| CONTROL-2 | 80 | 5102 | 14.9 | — |
| 2-Antegrade | 80 | 4419 | 10.35 | 13 |
| 2-Retrograde | 80 | 4144 | 3.28 | 19 |

As shown in the data in Table 1, each flow-modifying device reduces the rate of fluid flow in the retrograde direction by a greater amount than a fluid flow at the same pressure head in the antegrade direction. Table 2 shows the difference between the percent flow reduction in the retrograde direction and the percent flow reduction in the antegrade direction.

TABLE 2

| | | | | % retrograde flow |
|---|---|---|---|---|
| | Orifice | | | reduction − % |
| | Diameter | Inlet Diameter | Pressure Head | antegrade |
| Device | (mm) | (mm) | (mm Hg) | flow reduction |
| 1 | 5.5 | 11.0 | 2 | 1.7 |
| 2 | 6.0 | 13.2 | 2 | 2.5 |
| 1 | 5.5 | 11.0 | 10 | 1.7 |
| 2 | 6.0 | 13.2 | 10 | 1.8 |
| 1 | 5.5 | 11.0 | 50 | 4.8 |
| 2 | 6.0 | 13.2 | 50 | 2.4 |
| 1 | 5.5 | 11.0 | 80 | 3.3 |
| 2 | 6.0 | 13.2 | 80 | 5.4 |

Both flow-modifying devices (Device 1 and Device 2) reduced the fluid flow rate to a greater degree in the retrograde direction than in the antegrade direction. The amount of flow rate reduction varied as a function of the pressure head between 2 and 80 mm Hg. The increased amount of flow reduction in the retrograde direction varied from between about 1.7% and 5.4%, depending on the device configuration and the pressure head.

The invention includes other embodiments within the scope of the claims, and variations of all embodiments. Additional understanding of the invention can be obtained by referencing the detailed description of embodiments of the invention, below, and the appended drawings.

We claim:

1. A method of treating a venous valve-related condition by reducing blood flow in a retrograde direction away from a heart in a subject, the method comprising the steps of:
a. inserting a flow-modifying medical device into a body vessel within the deep venous system, the peripheral venous system or the perforating venous system, the flow-modifying medical device including a fluid flow channel extending longitudinally through the flow-modifying device between an inlet and an outlet and configured to restrict a rate of fluid flow therethrough in a retrograde direction from the outlet to the inlet more than a restriction of the rate of fluid flow through the fluid flow channel in an antegrade direction from the inlet to the outlet; and
b. deploying the flow-modifying medical device within the body vessel at a point of treatment to provide a first reduction in the rate of blood flow within the body vessel through the fluid flow channel in the retrograde direction away from the heart while providing a second reduction in the rate of blood flow within the body vessel through the fluid flow channel in the antegrade direction toward the heart that is less than the first reduction.

2. The method of claim 1, wherein the fluid flow channel is configured to restrict the rate of fluid flow therethrough by about 0.5-20% more in the retrograde direction than in the antegrade direction.

3. The method of claim 1, where the fluid flow channel extends along the longitudinal axis including an orifice positioned between the inlet having a first cross-sectional area and the outlet having a second cross-sectional area, the orifice having a third cross-sectional area that is less than the first cross-sectional area; the fluid flow channel being defined by an antegrade flow receiving surface extending from the inlet to the orifice, and a retrograde flow receiving surface extending from the outlet to the orifice, the antegrade flow receiving surface having a frustoconical cross section in a first radial bisecting plane containing the longitudinal axis.

4. The method of claim 3, wherein the retrograde flow receiving surface has an arcuate cross section in the first plane, the arcuate section having a surface with a first radius of curvature.

5. The method of claim 3, wherein the first reduction in the rate of blood flow in the retrograde direction is about 1-10% more in the second reduction in the rate of blood flow in the antegrade direction after positioning the medical device within the body vessel.

6. The method of claim 3, wherein antegrade flow receiving surface is oriented at an angle of about 20-70 degrees with respect to the longitudinal axis.

7. The method of claim 3, wherein the longitudinal axis passes through the center of the orifice.

8. The method of claim 3, wherein the ratio between the first cross-sectional surface area at the inlet and the third cross-sectional surface area at the orifice is between about 1.0 and 5.0.

9. The method of claim 8, wherein the ratio between the first cross-sectional surface area at the inlet and the third cross-sectional surface area at the orifice is between about 2.0 and 2.5.

10. The medical device of claim 3, wherein the third cross-sectional area of the orifice has a circular configuration with a diameter of at least 3 mm.

11. The method of claim 3, wherein at least a portion of the fluid flow channel is formed by a forming material comprising a biocompatible polyurethane.

12. The method of claim 11, wherein the medical device comprises a radially expandable support frame, and the forming material is attached to the support frame.

13. The method of claim 3, wherein at least a portion of the fluid flow channel is formed by a biodegradable material.

14. A method of treating a venous valve-related condition comprising the steps of:
a. inserting a flow-modifying medical device within a body vessel, the flow-modifying medical device having a longitudinal axis, the flow-modifying medical device comprising an annular bi-directional fluid flow restricting channel extending along the longitudinal axis from an inlet to an outlet, the fluid flow restricting channel including
i. an orifice having a thickness that is less than 10% of a maximum diameter of the orifice, the orifice being substantially parallel to and positioned longitudinally between the inlet and the outlet, the orifice having a first cross-sectional area oriented substantially perpendicular to the longitudinal axis and the inlet having a second cross-sectional area that is greater than the first cross-sectional area; and
ii. an antegrade flow receiving surface extending from the inlet to the orifice, joined at the orifice to a retrograde flow receiving surface extending from the orifice to the outlet, the antegrade flow receiving surface having a frustoconical cross section in a first hypothetical radial bisecting plane containing the longitudinal axis; the retrograde flow receiving surface having a curved cross section in the hypothetical first radial bisecting plane containing the longitudinal axis;
iii. the rate of fluid flow in an antegrade direction from the inlet through the flow restricting channel toward the outlet being reduced by less than the rate of fluid flow in a retrograde direction from the outlet through the flow restricting channel toward the inlet; and
b. deploying the flow-modifying medical device within the body vessel at a point of treatment to provide a first reduction in the rate of blood flow in the antegrade direction toward a heart that is less than a second reduction in the rate of blood flow in the retrograde direction away from the heart.

15. The method of claim 14, where the frustoconical cross section is angled between about 20° and 70° with respect to the longitudinal axis.

16. The method of claim 14, where the ratio of the second cross-sectional area of the inlet to the first cross-sectional area of the orifice is at least about 1.5.

17. The method of claim 14, wherein the inlet and the outlet each have a cross-sectional area of between about 75 mm² and 185 mm² and the ratio between a third cross-sectional area of the outlet and the first cross-sectional area of the orifice is between about 1 and 5.

18. The method of claim 14, wherein the orifice has a first cross-section having a circular configuration with a diameter of at least 3 mm.

19. The method of claim 14, where the flow-modifying medical device is further characterized in that:
a. the flow-modifying medical device includes a self-expanding implantable frame;
b. the retrograde flow receiving surface has an arcuate cross section in the first hypothetical plane;
c. the antegrade flow receiving surface is oriented at an angle of about 20°-45° with respect to the longitudinal axis,
d. the rate of fluid flow in the antegrade direction through the flow restricting channel is reduced by about 0.5-20% less than a reduction in the rate of fluid flow in the retrograde direction through the flow restricting channel;
e. the orifice contains the longitudinal axis and is configured as a circle having a diameter of at least 3 mm; and
f. the ratio between the second cross-sectional surface area of the inlet and the cross-sectional area of the first cross-sectional area of the orifice is between about 1.0 and 5.0.

20. A method of treating a venous valve-related condition comprising the steps of implanting a first flow-modifying medical device within a first vein and having a body vessel diameter; implanting a second flow-modifying medical device within a blood vessel in fluid flow communication with the first vein; the first flow-modifying medical device and the second flow-modifying medical device each including a fluid flow restricting channel extending between an inlet and an outlet and having a diameter that is less than the body vessel diameter, the flow-modifying medical device being configured to restrict the rate of fluid flow in an antegrade direction from the inlet to the outlet through the fluid flow restricting channel by less than a restriction of fluid flow in an retrograde direction from the outlet to the inlet through the fluid flow restricting channel, wherein
a. the minimum diameter of the flow restricting channel is an orifice having a first diameter that is substantially invariant to changes in fluid flow direction from the antegrade direction to the retrograde direction;
b. the fluid flow restricting channel includes an antegrade flow receiving surface extending form the inlet to the orifice and including an arcuate longitudinal cross section with a radius of curvature;
c. the ratio of the of the radius of curvature to the first diameter of the orifice being between about 0.5 and 0.8.
